(19) United States
(12) Patent Application Publication
(10) Pub. No.: US 2015/0083130 A1
(43) Pub. Date: Mar. 26, 2015

(54) START UP SELF-TESTING METHOD OF AIRWAY PRESSURE SENSOR FOR ANESTHESIA MACHINE

(71) Applicant: Beijing Aeonmed Co., Ltd., Beijing (CN)

(72) Inventor: Yaodong Zhang, Beijing (CN)

(21) Appl. No.: 14/396,312

(22) PCT Filed: Oct. 22, 2013
(86) PCT No.: PCT/CN2013/085725

§ 371 Date: Oct. 22, 2014

(30) Foreign Application Priority Data
Dec. 27, 2012 (CN) 201210580544.0

(51) Int. Cl. A61M 16/10; A61M 16/20; A61M 16/00

(52) U.S. Cl. CPC A61M 16/104; A61M 16/0003; A61M 16/0075; A61M 16/204; A61M 16/205; A61M 2016/0027; A61M 2202/0241; A61M 2205/3331; A61M 2205/702
USPC 128/203.28

(57) ABSTRACT

Disclosed is a starting up self-testing method of an airway pressure sensor for an anesthesia machine. In a manual state, a pressure value of the pressure sensor is detected, and after the detection, if the value is within an airway pressure measurement range but not within a sensor zero point voltage range, the anesthesia machine is switched to a machine-controlled state to detect the pressure value of the pressure sensor, so that under the machine-controlled state, an airway is in communication with a bellows to enable gas possibly left in the airway to flow into the bellows to thus change the pressure value in the airway, and hence if the current pressure value of the pressure sensor is reduced or directly in the sensor zero point voltage range, the pressure sensor is valid.

### TECHNICAL FIELD

The present disclosure relates to the technical field of anesthesia machines, and in particular, to a startup self-testing method of an airway pressure sensor for an anesthesia machine.

### BACKGROUND

An anesthesia machine is an advanced medical apparatus used to transfer various gases and volatile anesthetics, control and assist the respiration of a patient, and at the same time adjust the consciousness and pain sensation level of the patient during an operation.

As a medical apparatus for maintaining and supporting a life, the anesthesia machine can perform a startup self-testing, which is an indispensable step for guaranteeing the normal work of the anesthesia machine, and involves a plurality of testing items, including the testing of an airway pressure sensor, etc. At present, the startup self-testing of the airway pressure sensor of an anesthesia machine generally includes judging on a zero point of the pressure sensor, and if the zero-point voltage of the pressure sensor is within an allowable range, it is determined that the pressure sensor works normally; otherwise, it is determined that the pressure sensor is failed. Despite of its convenience, such a traditional self-testing method of an airway pressure sensor is defective in that, for example, when the zero-point voltage of the airway pressure sensor is beyond the allowable range, the pressure sensor itself might not be problematic, instead, it may be due to the presence of a certain pressure in the airway, which leads to the failure of the self-testing. In the sealed anesthesia system, some residual gas is left in the anesthesia machine after the previous usage of the anesthesia machine, and it takes much time for such residual gas to completely leak out, thus a certain pressure is present in the airway before the gas completely leaks out. Therefore, it is inaccurate to judge whether a pressure sensor is invalid only by the zero point of the pressure sensor.

### SUMMARY

Therefore, it is an object of the present disclosure to provide a startup self-testing method of an airway pressure sensor for an anesthesia machine. With the method, pressure values of the sensor are detected in both the manual state and the automatic state of the anesthesia machine, thereby improving the detection precision and the detection stability.

It is an object of the present disclosure to provide a startup self-testing method of an airway pressure sensor for an anesthesia machine. With the method, an anesthesia machine is directly switched from a manual state to an automatic state, and a pressure value of the pressure sensor is detected under the automatic state, so that the detection speed may be improved while ensuring the detection precision and stability.

To this end, the present disclosure employs the following technical solutions:

A startup self-testing method of an airway pressure sensor for an anesthesia machine includes:

Step a) of starting up an anesthesia machine, and sampling a pressure value of a pressure sensor arranged in the airway so that a pressure value of the pressure sensor V1 is obtained, wherein the started anesthesia machine is in a manual state in which a bellows and an airway in the anesthesia machine are insulated from each other;

Step b) of judging whether the pressure value V1 is within an airway pressure measurement range, and if so, performing Step c); otherwise, performing Step g);

Step c) of determining whether the pressure value V1 is within a zero-point voltage range of the pressure sensor, and if so, performing Step f); otherwise, performing Step d);

Step d) of switching the anesthesia machine from the manual state to an automatic state, in which the bellows is in communication with the airway of the anesthesia machine, and immediately sampling the pressure value of the pressure sensor in the current state after the switching from the manual state to the automatic state so that a pressure value V2 is obtained, and after time t1, sampling the pressure value of the pressure sensor again so that a pressure value V3 is obtained;

Step e) of judging whether the pressure value V3 is lower than the pressure value V2, and if so, performing Step f); otherwise, performing Step g);

Step f) of determining that the pressure sensor is valid, and ending the testing; and

Step g) of determining that the pressure sensor is invalid, and entering an inspection and repair state.

Preferably, in the startup self-testing method of an airway pressure sensor for an anesthesia machine, the airway pressure measurement range is from 2 KPa to 13 KPa.

Preferably, in the startup self-testing method of an airway pressure sensor for an anesthesia machine, the zero-point voltage range of the pressure sensor is from −0.5 KPa to 0.5 KPa.

Preferably, in the startup self-testing method of an airway pressure sensor for an anesthesia machine, the time t1 is in a range from 2 to 6 seconds.

A startup self-testing method of an airway pressure sensor for an anesthesia machine includes:

Step A) of starting up an anesthesia machine and switching the anesthesia machine from a manual state to an automatic state, wherein a bellows and an airway of the anesthesia machine are in communication with each other in the automatic state, and immediately sampling a pressure value of the pressure sensor in the current state after the switching from the manual state to the automatic state so that a pressure value V4 is obtained, and after time t2, sampling the pressure value of the pressure sensor again so that a pressure value V5 is obtained;

Step C) of judging whether the pressure value V5 is within a zero-point voltage range of the pressure sensor, and if so, performing Step E); otherwise, performing Step D;

Step D) of judging whether the pressure value V5 is lower than the pressure value V4, and if so, performing Step E); otherwise, performing Step F);

Step E) of determining that the pressure sensor is valid, and ending the testing; and

Step F) of determining that the pressure sensor is invalid, and entering an inspection and repair state.

Preferably, the startup self-testing method of an airway pressure sensor for an anesthesia machine further includes Step B) between Step A) and Step C), and Step B) includes judging whether the pressure value V5 is within an airway pressure measurement range, and if so, performing Step C); otherwise, performing Step F).

Preferably, in the startup self-testing method of an airway pressure sensor for an anesthesia machine, the airway pressure measurement range is from −2KPa to 13 KPa.

Preferably, in the startup self-testing method of an airway pressure sensor for an anesthesia machine, the zero-point voltage range of the pressure sensor is from −0.5 KPa to 0.5 KPa.

Preferably, in the startup self-testing method of an airway pressure sensor for an anesthesia machine, the time t2 is within a range from 5 to 10 seconds.

A use of the above startup self-testing method of an airway pressure sensor for an anesthesia machine in a valve is disclosed.

In comparison with the prior art, the invention has the following beneficial effects:

1) In the manual state, a pressure value of the pressure sensor is detected, and if the detected pressure value is within the airway pressure measurement range but beyond the zero-point voltage range of the pressure sensor, the anesthesia machine is switched to the automatic state to detect the pressure value of the pressure sensor again, so that under the automatic state, the airway is made in communication with the bellows to enable the gas possibly left in the airway to flow into the bellows, thereby changing the pressure in the airway. Therefore, if the current pressure value of the pressure sensor is lowered or directly falls into the zero-point voltage range of the pressure sensor at this time, it is indicated that the pressure sensor is valid, thus such a detection method improves the accuracy and stability of the detection process, and ensures the security and reliability of the pressure sensor.

2) By switching an anesthesia machine directly from the manual state to the automatic state after the anesthesia is powered on, the airway becomes in communication with the bellows under the automatic state to enable the gas possibly left in the airway to flow into the bellows, thereby the pressure in the airway is changed, and if a pressure value of the pressure sensor collected after a certain period of time is lower than a pressure value of the pressure sensor collected immediately after the switching, or if the pressure value of the pressure sensor collected after a certain period of time directly falls into the zero-point voltage range of the pressure sensor, it is indicated that the pressure sensor is valid, thus such a detection method not only ensures the accuracy and stability of the detection process to further ensure the security and reliability of the pressure sensor, but also simplifies the detection process, improves the detection speed, and lowers the detection difficulty.

3) The method of the invention may also be applied to the field of valves, to also achieve the above effect of detecting a pressure sensor, that is, the detection precision and stability can be guaranteed, and the security and reliability of a valve can be improved.

### BRIEF DESCRIPTION OF THE DRAWINGS

FIG. 1 is a flow chart showing a startup self-testing method of an airway pressure sensor for an anesthesia machine according to a first embodiment; and

FIG. 2 is a flow chart showing a startup self-testing method of an airway pressure sensor for an anesthesia machine according to a second embodiment.

### DETAILED DESCRIPTION OF THE EMBODIMENTS

### First Embodiment

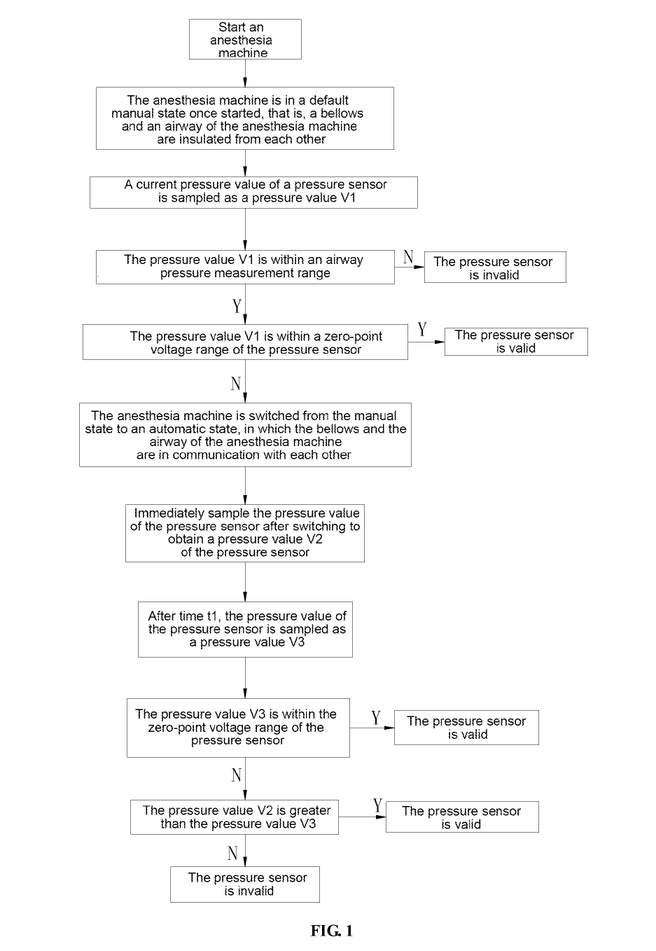

As shown in FIG. 1, a startup self-testing method of an airway pressure sensor for an anesthesia machine according to this embodiment includes the following steps:

Step 1) starting up an anesthesia machine, where the started anesthesia machine is by default in a manual state, in which a bellows and an airway in the anesthesia machine are insulated from each other.

Step 2) recording a pressure value of the pressure sensor arranged in the airway, so that a pressure value V1 of the pressure sensor is obtained.

Step 3) judging whether the pressure value V1 of the pressure sensor is within an airway pressure measurement range, which is from −2 KPa to 13 KPa in this embodiment, and if the pressure value V1 of the pressure sensor is within the airway pressure measurement range, Step 4) is performed; and if the pressure value V1 of the pressure sensor is beyond the airway pressure measurement range, the pressure sensor is invalid and needs for inspection and repair.

Step 4) judging whether the pressure value V1 of the pressure sensor is within a zero-point voltage range of the pressure sensor, which refers to an allowable range of zero-point voltage and is from 0.5 KPa to 0.5 KPa in this embodiment, and if the pressure value V1 of the pressure sensor is within the zero-point voltage range of the pressure sensor, the pressure sensor is valid, and the testing of the pressure sensor ends; and if the pressure value V1 of the pressure sensor is beyond the zero-point voltage range of the pressure sensor, Step 5) is performed for further testing.

Step 5) switching the anesthesia machine from the manual state to an automatic state, in which the bellows is in communication with the airway in the anesthesia machine and it is a default that an inspiration valve of the anesthesia machine is closed and an expiration valve of the anesthesia machine is open, and immediately sampling the pressure value of the pressure sensor in the current state after the switching from the manual state to the automatic state so that a pressure value V2 is obtained at this time, then after time t1, sampling the pressure value of the pressure sensor again so that a pressure value V3 is obtained at this time, where the time t1 is 3 seconds in this embodiment.

Step 6) judging whether the pressure value V3 of the pressure sensor is within the zero-point voltage range of the pressure sensor, and if the pressure value V3 of the pressure sensor is within the zero-point voltage range of the pressure sensor, it is indicated that the pressure sensor is valid, and the testing ends; and if the pressure value V3 of the pressure sensor is beyond the zero-point voltage range of the pressure sensor, Step 7) is performed for further testing.

Step 7) judging whether the pressure value V3 of the pressure sensor is less than the pressure value V2 of the pressure sensor, and if the pressure value V3 of the pressure sensor is less than the pressure value V2 of the pressure sensor, it is indicated that the pressure sensor is valid, and the testing ends; and if the pressure value V3 of the pressure sensor is no less than the pressure value V2 of the pressure sensor, it is indicated that the pressure sensor is invalid and needs for inspection and repair.

The above testing method employs the following principles that: initially the anesthesia machine is in the default manual state when powered on, the bellows and the airway of the anesthesia machine are insulated from each other in the manual state, and the bellows has a capacity over 1500 ml and is in communication with the ambient atmosphere, thus when the anesthesia machine is switched to the automatic state, the airway becomes in communication with the bellows, and a certain pressure is present in the airway if residual gas exists in the airway, and hence the gas will flow to the bellows, as a result, a pressure change occurs to the airway certainly; if such pressure change is detected by the airway pressure sensor, it is indicated that the pressure sensor is valid; or, if the pressure value of the pressure sensor in the automatic state is within the zero-point voltage range of the pressure sensor, it is also indicated that the pressure sensor is valid, otherwise, the pressure sensor is invalid and needs for repair, Therefore, if the pressure value of the pressure sensor as detected in the manual state is beyond the zero-point voltage range of the pressure sensor but within the airway pressure measurement range, the sensor is not necessarily invalid, because there might be the residual gas in the airway due to the fact that: in a sealed anesthesia system, some residual gas is left in the anesthesia machine after the previous usage of the anesthesia machine, and it takes much time for such residual gas to completely leak out, thus a certain pressure is present in the airway before the gas completely leaks out, thereby influencing the testing.

### Second Embodiment

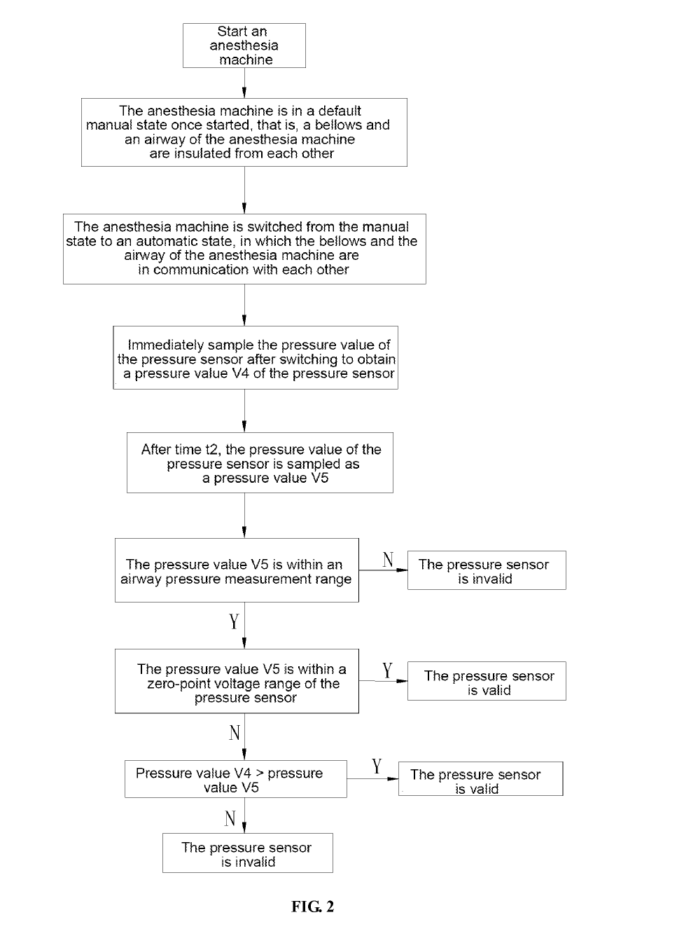

As shown in FIG. 2, a startup self-testing method of an airway pressure sensor for an anesthesia machine according to this embodiment includes the following steps.

Step 1) starting up an anesthesia machine, where the started anesthesia machine is by default in a manual state, in which a bellows and an airway in the anesthesia machine are insulated from each other.

Step 2) switching the anesthesia machine from the manual state to an automatic state, in which the bellows is in communication with the airway in the anesthesia machine and it is a default that an inspiration valve of the anesthesia machine is closed and an expiration valve of the anesthesia machine is open, and immediately sampling the pressure value of the pressure sensor in the current state after the switching from the manual state to the automatic state so that a pressure value V4 of the pressure sensor is obtained at this time, and after time t2, sampling the pressure value of the pressure sensor again so that a pressure value V5 of the pressure sensor is obtained at this time, where the time t2 is 6 seconds in this embodiment.

Step 3) judging whether the pressure value V5 of the pressure sensor is within an airway pressure measurement range, which is from −2 KPa to 13 KPa in this embodiment, and if the pressure value V5 of the pressure sensor is within the airway pressure measurement range, Step 4) is performed for further testing, and if the pressure value V5 of the pressure sensor is beyond the airway pressure measurement range, it is indicated that the pressure sensor is invalid and needs for inspection and repair.

Step 4) judging whether the pressure value V5 of the pressure sensor is in a zero-point voltage range of the pressure sensor, which is an allowable range of zero-point voltage and is from −0.5 KPa to 0.5 KPa in this embodiment, and if the pressure value V5 of the pressure sensor is within the zero-point voltage range of the pressure sensor, it is indicated that the pressure sensor is valid, and the testing ends; and if the pressure value V5 of the pressure sensor is beyond the zero-point voltage range of the pressure sensor, Step 5) is performed for further testing.

Step 5) judging whether the pressure value V5 of the pressure sensor is less than the pressure value V4 of the pressure sensor, and if the pressure value V5 of the pressure sensor is less than the pressure value V4 of the pressure sensor, it is indicated that the pressure sensor is valid, and the testing ends; and if the pressure value V5 of the pressure sensor is no less than the pressure value V4 of the pressure sensor, it is indicated that the pressure sensor is invalid and needs for inspection and repair.

The testing principles of this embodiment is consistent with those of the first embodiment, except that the detecting in the manual state is omitted, and the anesthesia machine is directly switched to the automatic state for detecting, thus not only achieving the detecting effect of the first embodiment, but also simplifying the detection processes, thus the detection difficulty may be lowered, and the detection speed may be improved.

The technical principles of the present disclosure have been described above in conjunction with specific embodiments. Such description is only used for explaining the principles of the present disclosure, rather than limiting the protection scope of the invention in any way. Based on such an explanation, other specific embodiments of the present invention may be made by one skilled in the art without creative work, and all these embodiments will fall into the protection scope of the invention.

1. A startup self-testing method of an airway pressure sensor for an anesthesia machine, comprising:
Step a) of starting up an anesthesia machine, and sampling a pressure value of a pressure sensor arranged in the airway so that a pressure value of the pressure sensor V1 is obtained, wherein the started anesthesia machine is in a manual state in which a bellows and an airway in the anesthesia machine are insulated from each other;
Step b) of judging whether the pressure value V1 is within an airway pressure measurement range, and if so, performing Step c); otherwise, performing Step g);
Step c) of determining whether the pressure value V1 is within a zero-point voltage range of the pressure sensor, and if so, performing Step f); otherwise, performing Step d);
Step d) of switching the anesthesia machine from the manual state to an automatic state, in which the bellows is in communication with the airway of the anesthesia machine, and immediately sampling the pressure value of the pressure sensor in the current state after the switching from the manual state to the automatic state so that a pressure value V2 is obtained, and after time t1, sampling the pressure value of the pressure sensor again so that a pressure value V3 is obtained;
Step e) of judging whether the pressure value V3 is lower than the pressure value V2, and if so, performing Step f); otherwise, performing Step g);
Step f) of determining that the pressure sensor is valid, and ending the testing; and
Step g) of determining that the pressure sensor is invalid, and entering an inspection and repair state.

2. The startup self-testing method of an airway pressure sensor for an anesthesia machine according to claim 1, wherein the airway pressure measurement range is from 2 KPa to 13 KPa.

3. The startup self-testing method of an airway pressure sensor for an anesthesia machine according to claim 1, wherein the zero-point voltage range of the pressure sensor is from −0.5 KPa to 0.5 KPa.

4. The startup self-testing method of an airway pressure sensor for an anesthesia machine according to claim 1, wherein the time t1 is in a range from 2 to 6 seconds.

5. A startup self-testing method of an airway pressure sensor for an anesthesia machine, comprising:
Step A) of starting up an anesthesia machine and switching the anesthesia machine from a manual state to an automatic state, wherein a bellows and an airway of the anesthesia machine are in communication with each other in the automatic state, and immediately sampling a pressure value of the pressure sensor in the current state after the switching from the manual state to the automatic state so that a pressure value V4 is obtained, and after time t2, sampling the pressure value of the pressure sensor again so that a pressure value V5 is obtained;
Step C) of judging whether the pressure value V5 is within a zero-point voltage range of the pressure sensor, and if so, performing Step E); otherwise, performing Step D;
Step D) of judging whether the pressure value V5 is lower than the pressure value V4, and if so, performing Step E); otherwise, performing Step F);
Step E) of determining that the pressure sensor is valid, and ending the testing; and
Step F) of determining that the pressure sensor is invalid, and entering an inspection and repair state.

6. The startup self-testing method of an airway pressure sensor for an anesthesia machine according to claim 5, further comprising, between Step A) and Step C), Step B) of judging whether the pressure value V5 is within an airway pressure measurement range, and if so, performing Step C); otherwise, performing Step F).

7. The startup self-testing method of an airway pressure sensor for an anesthesia machine according to claim 6, wherein the airway pressure measurement range is from −2 KPa to 13 KPa.

8. The startup self-testing method of an airway pressure sensor for an anesthesia machine according to claim 5, wherein the zero-point voltage range of the pressure sensor is from −0.5 KPa to 0.5 KPa.

9. The startup self-testing method of an airway pressure sensor for an anesthesia machine according to claim 5, wherein the time t2 is within a range from 5 to 10 seconds.

10. A use of the startup self-testing method of an airway pressure sensor for an anesthesia machine according to claim 1 in a valve.
